(19) United States
(12) Patent Application Publication
(10) Pub. No.: US 2010/0228081 A1
(43) Pub. Date: Sep. 9, 2010

(54) BIO-FEEDBACK EXERCISE METHOD FOR MEN'S SEXUAL DISORDERS

(76) Inventors: Eugene Mikhailenok, San Diego, CA (US); Olga Voronina, San Diego, CA (US); Olga Potapova, San Francisco, CA (US); Andrey Belayev, San Francisco, CA (US)

Correspondence Address: FRANK G MORKUNAS, P.O. Box 601662, SAN DIEGO, CA, 92160, US

(21) Appl. No.: 12/713,933

(22) Filed: Feb. 26, 2010

Related U.S. Application Data
(60) Provisional application No. 61/157,252, filed on Mar. 4, 2009.

(51) Int. Cl. A61F 5/00

(52) U.S. Cl. 600/38

(57) ABSTRACT

A method of identifying, toning, and strengthening a specific muscle group comprising the bulbospongiosus and the ishiocavernosus muscles in an attempt to alleviate or overcome men's sexual disorders. A muscle-sensor is attached to the muscle group of a user and to a computing device in communication with a display device. As the user contracts and relaxes the muscle group, the contractions and relaxations are displayed in real-time on the display device. Such real-time display teaches the user to identify how to identify and exercise the muscle group and to set a goal as to how long the user is to hold the contraction. An external emotionally significant motivational visual stimulus may be programmed into the computing device to display the visual stimulus only when the goal as to the contraction is attained and maintained.

### CROSS REFERENCES TO RELATED APPLICATIONS

This application claims the benefit of U.S. Provisional Application No. 61/157,252, filed on Mar. 4, 2009.

### STATEMENT REGARDING FEDERALLY-SPONSORED RESEARCH OR DEVELOPMENT

Not applicable.

### BACKGROUND

This method relates to an improvement in exercise methods involving the bulbospongiosus muscle and the ishiocavernosus muscle, both of which are located in the scrotum area, and more particularly to a real-time visual and bio-feedback of the results of one's exercise of these muscles with a view toward attaining a pre-set goal and modifying one's technique based on the real-time feedback being provided and thereby enhance one's sexual function and experiences.

Our method replaces the prior art methods of randomly contracting and relaxing the respective muscles with an individualized approach based on objectively [physically] based on real-time visual feedback signals. These visual signals inform each person of their specific conditions and variations in their physiological functions. From immediate feedback, individualized to a specific person, that person will progress more rapidly in the strengthening and toning of the bulbospongiosus and the ishiocavernosus muscles. The introduction of the visual feedback signal to the exercise process enables the person to progress at his own level based on individualized time, need, and desire.

The foregoing has outlined some of the more pertinent objects of the bio-feedback muscle-exercise method. These objects should be construed to be merely illustrative of some of the more prominent features and applications of the bio-feedback Muscle-exercise method. Many other beneficial results can be attained by applying the disclosed bio-feedback muscle-exercise method in a different manner or by modifying the bio-feedback muscle-exercise method within the scope of the disclosure. Accordingly, other objects and a fuller understanding of the bio-feedback muscle-exercise method may be had by referring to the summary of the bio-feedback muscle-exercise method and the detailed description of the preferred embodiment in addition to the scope of the bio-feedback muscle-exercise method defined by the claims taken in conjunction with the accompanying drawings.

### SUMMARY

The above-noted problems, among others, are overcome by the bio-feedback muscle-exercise method. Briefly stated, the bio-feedback muscle-exercise method contemplates a real-time visual and bio-feedback of the results of one's exercises while engaging in the specific exercises designed to relieve or minimize a male's sexual dysfunction or enhance one's sexual experience or both. A pre-set goal is inputted into a computer, muscle sensor which is capable of emitting and conveying electromyograms [EMGs] to a visual display is strategically placed on a user which involve the requisite muscles [the bulbospongiosus muscle and the ishiocavernosus muscle] with the sensors being in direct communication with the computer. As the exercise program begins, the sensor displays to the user that user's performance as compared to the pre-set goal with a view toward modifying the exercises based on the real-time feedback being provided in continued efforts to attain that pre-set goal. The bulbospongiosus muscle and the ishiocavernosus muscle are very close to one another thereby generally making it difficult to distinguish the EMG of one as contrasted or compared to the other by means of the skin EMG sensor. To successfully relieve or minimize one's sexual disorder, these two muscles both should be strong and toned. Consequently, both are exercised simultaneously without segregating or differentiating one from the other.

The foregoing has outlined the more pertinent and important features of the bio-feedback muscle-exercise method for treating male sexual disorders in order that the detailed description that follows may be better understood so the present contributions to the art may be more fully appreciated. Additional features of the bio-feedback muscle-exercise method for treating male sexual disorders will be described hereinafter which will form the subject of the claims. It should be appreciated by those skilled in the art that the conception and the disclosed specific embodiment may be readily utilized as a basis for modifying or designing other structures and methods for carrying out the same purposes of the bio-feedback muscle-exercise method for treating male sexual disorders. It also should be realized by those skilled in the art that such equivalent constructions and methods do not depart from the spirit and scope of the bio-feedback muscle-exercise method for treating male sexual disorders as will be set forth in the claims.

### BRIEF DESCRIPTION OF THE DRAWINGS

For a fuller understanding of the nature and objects of the bio-feedback muscle-exercise method for treating male sexual disorders, reference should be had to the following detailed description taken in conjunction with the accompanying drawings

FIGS. 1 and 2 are graphs illustrating a sample display of a person's goal and actual performance in relation to that goal.

FIG. 3 is a graph of a person's goal and actual performance tied to a visual motivational stimulus.

### DETAILED DESCRIPTION

The method involved in our bio-feedback approach currently involves two muscles which are extremely important to a man's sexual functions and experiences [the bulbospongiosus muscle and the ishiocavernosus muscle each of which are under the scrotum], measuring and displaying the results of the exercises for these muscles while a person is engaged in the muscle exercise [feedback], and having that person adjust their contraction and relaxation rates, as well as the strength of the contraction, in an attempt to achieve a pre-set goal as established by or for that person and individualized to that person.

The method includes a computer programmed algorithm and a computerized strategy individualized to the particular person. A sensor in communication with the computer and monitor is placed on the person's body on the projections of the bulbospongiosus muscle and the ishiocavernosus muscle. This sensor is a conventionally available sensor suited for the intended purpose. A typical conventionally available sensor for both muscles could be, but is not limited to, a FlexComp/Myotrac, model number TTL SA9503M; or a MyoScan EMG Sensor, model number T9503M).

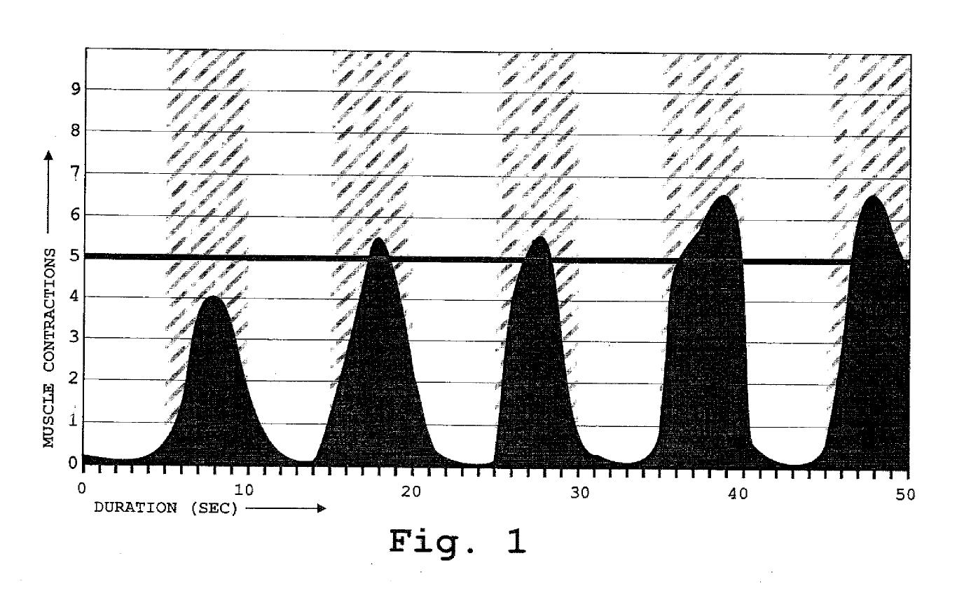

Once the sensor is properly placed, the person may set a goal for himself to achieve by inputting such into the computer. When the person begins exercising, the sensor registers information about the strength of the contractions and their duration, and the relaxations and their duration. The person's display monitor would display a graph similar to what is illustrated in FIG. 1 but only with the Goal [solid line on the graph] being displayed at that particular time.

The method involved in this bio-feedback approach consists of two stages. The first stage Goal is to help the person identify the bulbospongiosus and the ishiocavernosus muscles and acquire initial skills in contracting them. This stage is required because the bulbospongiosus and the ishiocavernosus muscles participate in visceral reflex and are not intended to be contracted voluntary. However power, tone, and function of the bulbospongiosus and the ishiocavernosus muscles can be developed only by means of their voluntary contraction. During the first stage of training the person is trying to achieve the goal depicted on the graph (FIG. 1).

The bottom horizontal axis of the graph depicts duration typically in seconds (50 seconds on this graph). This axis of duration is depicted in intervals of one-second each. The areas hatched represent periods of time when the person should contract these two muscles and hold that contraction. The non-hatched intervals represent periods of time when the person will relax these muscles. Typically the hatched areas should be colored so that the periods for contraction are more easily discerned by the user.

The vertical axis represents the level or degree of contraction of the bulbospongiosus and the ishiocavernosus muscles. The solid horizontal line depicts the level of the bulbospongiosus and the ishiocavernosus muscles contraction which the person is attempting to achieve during the exercise and training (the Goal). The person can move this line along the vertical axis thus making the task more difficult by moving that line up, or making the exercise task easier by moving the line down. Positioning of this line depends on the user's personal and individual ability of to contract the bulbospongiosus and the ishiocavernosus muscles.

The person's display monitor would display a graph similar to what is illustrated in FIG. 1 but only with the Goal [solid horizontal line on the graph] being displayed at that particular time.

By this bio-feedback method, the person is given immediate feedback, in real-time, as to how that person is doing and/or progressing and what adjustment it may take to attain the individualized Goal. With the visual feedback presented to the person, the person can adjust the muscle contractions, durations, and relaxions to come closer to and finally comport with that person's pre-set Goal. Once the Goal is reached the person may adjust the Goal to a higher plane for even greater results and proceed with the exercises until the next Goal has been attained. The re-set of the Goal can continue for as long as the person desires or reaches that person's final pre-set Goal unique to that person.

Once the Goal is reached the person can proceed to the second stage of the training. The Goal of this stage is to teach the person the skill of prolonged contraction of the bulbospongiosus and the ishiocavernosus muscles to increase their strength and toning. Prolonged muscle contraction is a very difficult task. It requires one's voluntary effort and may cause negative reactions and emotions.

A unique feature of this stage of the training is that the person uses the graph to pre-set the Goal [similar to as done during the first stage] but direct feedback is provided by means of an external motivational stimuli such as a movie or slide show being displayed on the monitor to be seen in real-time by the user as the user engages in the exercise routines instead of the graph-type display. We have found that such external displays have a positive motivational effects to and on the user. The use of emotionally significant motivational stimulus is a significant difference as compared to existing biofeedback methods of training. This form of stimulus distracts the person from negative feelings which result from intensive and hard training and makes achievement of the goal more pleasurable and desirable.

A sensor in communication with the computer and the monitor is placed on the person's body on the projection of bulbospongiosus and ishiocavernosus muscles, which as previously indicated, are under the scrotum area, much in the same manner as was done during stage 1 of the training.

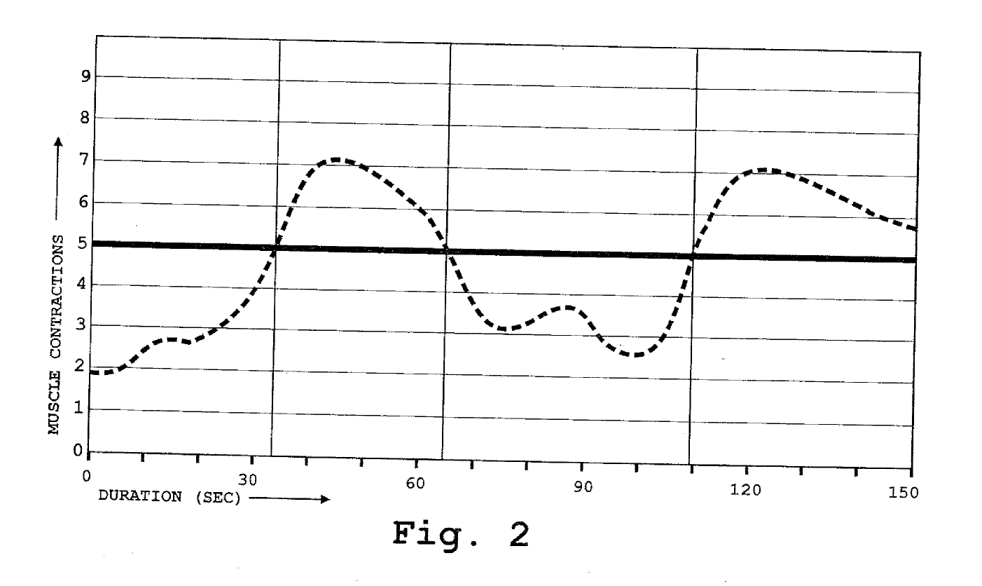

FIG. 2 is a graph illustrating a sample display of a person's pre-set goal at the second stage of the training. The solid horizontal line depicts the level of the bulbospongiosus and the ishiocavernosus muscles contraction, which the person is attempting achieve during the training (i.e., the Goal). The person can move this line along the vertical axis thus making the exercise task more difficult (moving the Goal line up) or move the Goal line down thereby simplifying the exercise task. Positioning this line will be dependent on the individual's ability to contract the bulbospongiosus and the ishiocavernosus muscles during the exercises. Results are conveyed to the user on the monitor screen, In FIG. 2, the user's real-time results are illustrated in dashed line.

After the Goal for the user is established the graph approach may be replaced with a video, a slide show, or a movie of the user's chosing [referred to as motivitaional stimuli]. The screen [monitor] then will display to the user the selected motivational stimuli as the user engages in the exercises. It has been found that once a user attains this level, the exercise technique is more on the sub-conscious plane rather than on the conscious plane.

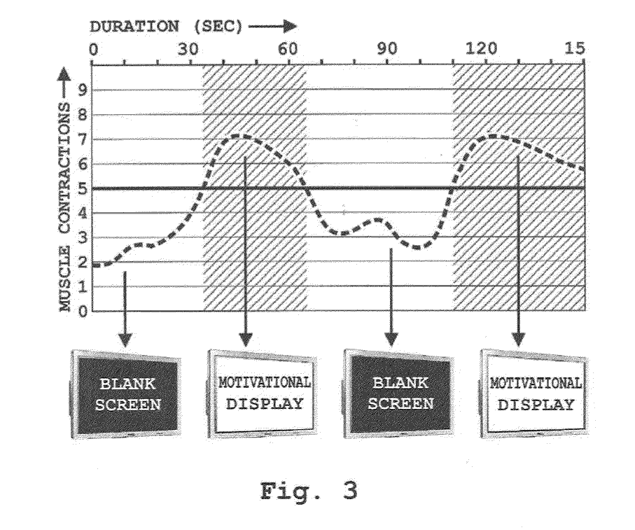

FIG. 3 illustrates a sample displays monitor screens during the second stage of the training. The person can watch the movie/slides only on the condition of correct contraction of the bulbospongiosus and the ishiocavernosus muscles. Correct contraction means that contraction of the bulbospongiosus and the ishiocavernosus muscles is supported on the level pre-set by the person or higher level.

Demonstration of the movie/slide-show is automatically stopped and the screen or monitor becomes blank if the level of contraction of the bulbospongiosus and the ishiocavernosus muscles does not reach or becomes lower than the level pre-set.

Because the user is motivated with a desire to watch movie/slide-show, the user increases the contractions of the bulbospongiosus and the ishiocavernosus muscles and holds that contraction for a prolonged, the requisite, period of time. The duration of the training session depends on the fatigue of the person but typically is not more then 30 minutes.

Once the Goal is reached the person may adjust the Goal to a higher plane for even greater results and proceed with the exercises until the next Goal has been attained. The re-set of the Goal can continue for as long as the person desires or reaches that person's final pre-set Goal unique to that person.

The software program to facilitate this method is structured on pre-set information about the optimum correlation between duration of the contraction and relaxation periods and is synchronized to compare the optimums with the person's actual muscle-contraction phases during the exercises.

The software program records and displays the results. Given these results, the person makes attempts to adjust the exercises to achieve the goals. The displayed results gives the person an objective reference as well as full control over the quality of the exercise fulfillment.

Although this bio-feedback muscle-exercise method of muscle training for overcoming men's sexual disorders has been described in its preferred forms with a certain degree of particularity, it is understood that the present disclosure of the preferred forms has been made only by way of example and numerous changes in the details of the method steps may be resorted to without departing from the spirit and scope of the bio-feedback muscle-exercise method. Accordingly, the scope of the bio-feedback muscle-exercise method should be determined not by the embodiment[s] illustrated, but by the claims to be provided later and their legal equivalents.

Applicant[s] have attempted to disclose all the embodiment[s] of the bio-feedback muscle-exercise method for overcoming men's sexual disorders that could be reasonably foreseen. It must be understood, however, that there may be unforeseeable insubstantial modifications to bio-feedback muscle-exercise method that remain as equivalents and thereby falling within the scope of the bio-feedback muscle-exercise method.

We claim:

1. A method of overcoming men's sexual disorders by strengthening a muscle group comprising a bulbospongiosus muscle and a ishiocavernosus muscle, said method comprising the steps of:
(a) attaching a muscle-contraction sensor to said muscle group of a person and conveying to a visual display device said person's muscle group contraction rate as to duration of contracting and relaxing said muscle group as detected by said muscle-contraction sensor;
(b) establishing a goal as to duration of said relaxing and contracting of said muscle group and conveying said goal to a computing device which is in communication with said visual display device; and
(c) commencing to exercise said muscle group by said contracting and relaxing said muscle group to attain said goal.

2. The method of claim 1 further comprising viewing said visual display device to see real-time results of said exercise as to properly identifying and exercising said muscle group and attempting to attain said goal.

3. The method of claim 1 wherein after said goal is attained, adjusting said goal and repeating the method steps of claim 1 as often as desired.

4. The method of claim 1 further comprising the step introducing an external visual stimulus in communication with said computing device wherein said external visual stimulus is displayed to said visual device for viewing by said person only when said goal is attained.

5. The method of claim 1 further comprising the step introducing an external visual stimulus in communication with said computing device wherein said external visual stimulus is not displayed to said visual device for viewing by said person until said goal is attained and maintained.
